(19) United States
(12) Patent Application Publication
(10) Pub. No.: US 2024/0260823 A1
(43) Pub. Date: Aug. 8, 2024

(54) SYSTEMS AND METHODS FOR THE IDENTIFICATION, EVALUATION, AND/OR CLOSED-LOOP REPROCESSING OF LUMENS

(71) Applicant: Saban Ventures Pty Limited, Macquarie Park, NSW (AU)

(72) Inventors: Gavin SPARGO, Box Hill, NSW (AU); Amir Hossein KAYVANPOUR, Carlingford, NSW (AU); Mohsen NABIPOOR, Macquarie Park, NSW (AU)

(21) Appl. No.: 18/566,359

(22) PCT Filed: Jun. 9, 2022
(86) PCT No.: PCT/AU2022/050567

§ 371 Date: Dec. 1, 2023

(30) Foreign Application Priority Data
Jun. 9, 2021 (AU) 2021901734

(51) Int. Cl. A61B 1/12; A61B 1/00

(52) U.S. Cl. CPC A61B 1/125; A61B 1/00006

(57) ABSTRACT

Systems and methods for identifying a fluidic configuration of a medical device having at least one lumen, identifying at least one lumen of a medical device, evaluating the integrity of a lumen of a medical device, and reprocessing a lumen of a medical device are presented. For example, these methods include flowing a fluid comprising a known specific gravity through the lumen, measuring the flow rate and/or pressure differential of the fluid being flowed through the lumen, and computing the flow coefficient of the lumen. The flow rate, pressure differential and/or computed flow coefficient may then be compared to known parameters for medical devices and their respective lumens to identify the medical device, each lumen, detect any faults, and/or irrigate the lumen with a fluid composition thereby reprocessing the medical device.

### CROSS REFERENCE TO RELATED APPLICATIONS

This application claims the benefit of Australian provisional application number 2021901734, filed on Jun. 9, 2021, the entirety of which is incorporated by reference herein.

### BACKGROUND

Any discussion of the prior art throughout the specification should in no way be considered as an admission that such prior art is widely known or forms part of common general knowledge in the field.

An endoscope is an elongate tubular medical device that may be rigid or flexible and which incorporates an optical or video system and light source. Typically, an endoscope is configured so that one end can be inserted into the body of a patient via a surgical incision or via one of the natural openings of the body. Internal structures near the inserted end of the endoscope can thus be viewed by an external observer.

As well as being used for investigation, endoscopes are also used to carry out diagnostic and surgical procedures. Endoscopic procedures are increasingly popular as they are minimally invasive in nature and provide a better patient outcome (through reduced healing time and exposure to infection) enabling hospitals and clinics to achieve higher patient turnover.

Endoscopes typically take the form of a long tube-like structure with a ‘distal tip’ at one end for insertion into a patient and a ‘connector end’ at the other end, with a control handle at the center of the length. The connector end is normally hooked up to a supply of light, water, suction and pressurized air. The control handle is held by the operator during the procedure to control the endoscope via valves and control wheels. The distal tip contains the camera lens, lighting, nozzle exits for air and water, exit point for suction and forceps. All endoscopes have internal channels used either for delivering air and/or water, providing suction or allowing access for forceps and other medical equipment required during the procedure. Some of these internal channels run from one end of the endoscope to the other, while others run via valve sockets at the control handle. Some channels bifurcate while and others join from two into one.

The high cost of endoscopes means they must be re-used. As a result, because of the need to avoid cross infection from one patient to the next, each endoscope must be reprocessed (e.g., thoroughly cleaned, disinfected, sterilized, and/or tested for leaks) after each use. This involves the cleaning of not only the outer of the endoscope, but also cleaning and disinfecting the internal channels/lumens.

Endoscopes used for colonoscopy procedures are typically between 2.5 and 4 meters long and have one or more lumen channels of diameter of no more than a few millimeters. Ensuring that such long narrow channels are properly cleaned and disinfected between patients presents a considerable challenge. The challenge of cleaning is also made more difficult by the fact that there is not just one configuration/type of endoscope. Indeed, there are a variety of endoscopic devices, each suited to a particular insertion application i.e. colonoscopes inserted into the colon, bronchoscopes inserted into the airways and gastroscopes for investigation of the stomach. Gastroscopes, for instance, are smaller in diameter than colonoscopes; bronchoscopes are smaller again and shorter in length while duodenoscopes have a different tip design to access the bile duct.

A variety of options are available to mechanically remove biological residues from the lumen which is the first stage in the cleaning and disinfection process. By far the most common procedure for cleaning the lumens utilize small brushes mounted on long, thin, flexible lines. Brushing is the mandated means of cleaning the lumen in some countries. These brushes are fed into the lumens while the endoscope is submerged in warm water and a cleaning solution. The brushes are then pushed/pulled through the length of the lumens in an effort to scrub off the soil/bio burden. Manual back and forth scrubbing is typically required. Water and cleaning solutions are then flushed down the lumens. These flush-brush processes are repeated three times or until the endoscope reprocessing technician is satisfied that the lumen is clean. At the end of this cleaning process air is pumped down the lumens to dry them. A flexible pull-through device having wiping blades may also be used to physically remove material. A liquid flow through the lumen at limited pressure can also be used.

In general, however, only the larger suction/biopsy lumens can be cleaned by brushing or pull-throughs. Air/water channels are too small for brushes so these lumens are usually only flushed with water and cleaning solution.

After mechanical cleaning, a chemical clean is carried out to remove the remaining biological contaminants. Because endoscopes are sensitive and expensive medical instruments, the biological residues cannot be treated at high temperatures or with strong chemicals. For this reason, the mechanical cleaning needs to be as thorough as possible. In many cases, the current mechanical cleaning methodologies fail to fully remove biofilm from lumens, particularly where cleaning relies on liquid flow alone. Regardless of how good the conventional cleaning processes are, it is almost inevitable that a small microbial load will remain in the channel of the lumen.

There has been significant research to show that the method of cleaning with brushes, even when performed as prescribed, does not completely remove biofilm in endoscope lumens. As well as lacking in efficacy, the current manual brushing procedures suffer from other drawbacks. The large number of different endoscope manufacturers and models results in many minor variations of the manual cleaning procedure. This has led to confusion and ultimately poor compliance in cleaning processes. The current system of brushing is also hazardous in that the chemicals that are currently used to clean endoscopes can adversely affect the reprocessing staff.

The current system of manual brushing is also labor intensive, leading to increased cost. Thus, the current approaches to cleaning and disinfecting the lumens in medical cleaning apparatus are still inadequate and residual microorganisms are now recognized as a significant threat to patients and staff exposed to these devices.

There is evidence of bacterial transmission between patients from inadequate cleaning and disinfection of internal structures of endoscopes which in turn has led to patients acquiring mortal infections. Between 2010 and 2015 more than 41 hospitals worldwide, most in the U.S., reported bacterial infections linked affecting 300 to 350 patients to the scopes, (http://www.modernhealthcare.com/article/20160415/NEWS/160419937). It would be expected that a reduction in the bioburden in various medical devices would produce a concomitant overall reduction in infection rates and mortality. In addition, if endoscopes are not properly cleaned and dried, biofilm can build up on the lumen wall. Biofilms start to form when a free-floating microorganism attaches itself to a surface and surrounds itself with a protective polysaccharide layer. The microorganism then multiplies, or begins to form aggregates with other microorganisms, increasing the extent of the polysaccharide layer. Multiple sites of attachment can in time join up, forming significant deposits of biofilm. Once bacteria or other microorganisms are incorporated in a biofilm, they become significantly more resistant to chemical and mechanical cleaning than they would be in their free-floating state. The organisms themselves are not inherently more resistant, rather, resistance is conferred by the polysaccharide film and the fact that microorganisms can be deeply embedded in the film and isolated from any chemical interaction. Any residual biofilm remaining after an attempt at cleaning quickly returns to an equilibrium state and further growth of microorganisms within the film continues. Endoscopes lumens are particularly prone to biofilm formation. They are exposed to significant amounts of bioburden, and subsequent cleaning of the long narrow lumens is quite difficult due to inaccessibility and the inability to monitor the cleaning process.

There is considerable pressure in medical facilities to reprocess endoscopes as quickly as possible. Because endoscopes are cleaned by hand, training and attitude of the technician are important in determining the cleanliness of the device. Residual biofilm on instruments can result in a patient acquiring an endoscope acquired infection. Typically, these infections occur as outbreaks and can have fatal consequences for patients.

There remains a need to overcome or ameliorate at least one of the disadvantages of the prior art, or to provide a useful alternative.

### BRIEF SUMMARY

According to a first aspect of the present invention, there is provided a method of identifying a fluidic configuration of a medical device having at least one lumen comprising:
determining the fluidic resistance of the at least one lumen of the medical device; and
identifying the fluidic configuration based on the determined fluidic resistance of at least one lumen.

According to a second aspect of the present invention, there is provided a method of identifying at least one lumen of a medical device comprising:
determining the fluidic resistance of the at least one lumen of the medical device; and
identifying the at least one lumen of the medical device based on at least its respective determined fluidic resistance.

According to a third aspect of the present invention, there is provided a method of evaluating the integrity of a lumen of a medical device comprising:
determining a fluidic resistance of the lumen; and
comparing the fluidic resistance of the lumen to a known nominal range of fluidic resistance values of the lumen.

According to a fourth aspect of the present invention, there is provided a method of cleaning a lumen of a medical device comprising:
determining a fluidic resistance of the lumen; and
flowing a fluid through the lumen based on the determined fluidic resistance.

According to a fifth aspect of the present invention, there is provided a method of identifying a fluidic configuration of a medical device having at least one lumen comprising:
measuring a fluidic parameter of the at least one lumen of the medical device; and
identifying the fluidic configuration based on the measured fluidic parameter of at least one lumen.

According to a sixth aspect of the present invention, there is provided a method of identifying at least one lumen of a medical device comprising:
measuring a fluidic parameter of the at least one lumen of the medical device; and
identifying the at least one lumen of the medical device based on the measured fluidic parameter of the at least one lumen.

According to a seventh aspect of the present invention, there is provided a method of evaluating the integrity of a lumen of a medical device comprising:
measuring a fluidic parameter of the at least one lumen of the medical device; and
evaluating the integrity of the lumen of the medical device based on the measured fluidic parameter of at least one lumen.

According to an eighth aspect of the present invention, there is provided a method of reprocessing a lumen of a medical device comprising:
measuring a the fluidic parameter of the at least one lumen of the medical device; and
reprocessing the lumen of the medical device based on the measured fluidic parameter of at least one lumen.

According to a ninth aspect of the present invention, there is provided a method of calibrating a medical device reprocessing system comprising:
flowing a first fluid through a lumen of the medical device;
measuring a first pressure and a first flow rate of the first fluid;
stopping the flow of the first fluid;
introducing a dose of a second fluid into the lumen of the medical device;
   wherein the viscosity of the second fluid is greater than the viscosity of the first fluid;
flowing the first fluid to move the second fluid through the lumen of the medical device and measuring a second flow rate of the first fluid; and
using the first and second flow rates to inform the operating parameters of the medical device reprocessing system in the reprocessing of the lumen of the medical device.

Therefore, the disclosure provides systems and methods for the identification, evaluation, and/or closed-loop reprocessing of lumens of medical devices; this can solve various problems in the art. For example, when an endoscope is connected to an automated reprocessing device it can be beneficial for the device to detect which configuration of endoscope is connected, so that the correct cleaning/disinfection parameters can be used for that particular endoscope configuration. For example, there are different makes/models of endoscopes, which can each be characterized by different configurations. For instance, the endoscopes may have different flow paths, geometries, lumen geometries, etc. As can be appreciated, this is also true of various medical devices that contain lumens.

Accordingly, some aspects of the disclosed methods comprise measuring/determining a fluidic parameter (e.g., flow rate, pressure, flow coefficient, and/or fluidic resistance) of the at least one lumen of the medical device. Measuring/determining the fluidic parameter of at least one lumen of the medical device may comprise flowing a fluid through the at least one lumen and measuring a flow rate and/or a pressure differential of the fluid being flowed through the at least one lumen. In some embodiments, measuring/determining the fluidic parameter may comprise calculating a flow coefficient. The fluid parameter may then be used beneficially, e.g., to identify the lumen, to evaluate it, and/or to facilitate its cleaning.

In one embodiment, the flow coefficient of the at least one lumen may be computed from the flow rate and the pressure differential. Upon comparison of the computed flow coefficient of the at least one lumen with a database that comprises a list of medical devices and associated flow coefficients(s) for its respective lumen(s), the fluidic configuration of the medical device may be identified, the lumen of the medical device may be identified, the integrity of the lumen may be evaluated, and/or medical device may be reprocessed.

Reprocessing parameters may be selected based on the fluidic parameter or flow coefficient, including controlling an amount of cleaning, a volume, a dose, a number of shots, a timing of each shot of the number of shots, and a velocity of the fluid composition. As used herein the term “dose” mean a quantity of fluid flowed through one or more lumens of the medical device. In some cases the dose may fill the entire lumen, and in other cases, the dose may comprise less than the entire volume of the lumen and may be referred to as either a “dose or a “shot” interchangeably. Suitable velocities can be on the order of 1000 mm/s. Of course, it should be appreciated that any suitable velocity can be implemented in accordance with embodiments of the disclosure.

In some embodiments, the fluid comprises water and sodium bicarbonate. For example, between 180-200 grams of sodium bicarbonate may be used to reprocess a medical device, e.g., a flexible gastrointestinal endoscope. For one of the larger channels (e.g. suction/biopsy channels), 80-100 grams may be used. For one of the smaller channels (e.g. air/water channels) 60-80 grams may be used, and for some of the smallest channels (e.g. auxiliary channels) 10-20 grams may be used. More details around the systems/methods for reprocessing lumens using fluidic compositions comprising one or more cleaning agents can be seen in Applicant's concurrently filed patent application titled, “Systems and Methods for Cleaning Lumens with Fluidic Compositions,” which claims priority to Australian provisional patent application number 2021901729, filed Jun. 9, 2021. The contents of these applications are hereby incorporated by reference, in their entirety, especially as it relates to systems and methods for reprocessing medical devices having lumens using fluidic compositions comprising one or more cleaning agents.

As an example, a reprocessing cycle may comprise: 1 shot in the auxiliary lumen, 9 shots in the suction biopsy lumen, 1 shot in the auxiliary lumen, 3 shots in in the suction biopsy lumen, 1 shot in the auxiliary lumen, and 9 shots in the suction biopsy lumen. In parallel, 6 shots may be delivered to the air lumen, and 6 shots delivered to the water lumen.

Timing for each shot may vary between about 15 seconds between each shot to the larger (e.g. suction/biopsy) lumens to about 30 seconds between each shot to the smaller (e.g. air/water and auxiliary) lumens.

In further embodiments, a method of closed-loop reprocessing of a medical device comprises: first, calibrating the reprocessing system to at least one lumen of the medical device and second, using this calibration to reprocess the at least one lumen of the medical device in a closed-loop manner.

Thus, in some embodiments, a method of calibrating a medical device reprocessing system comprises: flowing air through a lumen of the medical device; measuring a first air pressure and a first air flow rate; stopping the step of flowing air through the lumen of the medical device; flowing a dose of water through the lumen of the medical device; flowing air though the lumen of the medical device and measuring a second air pressure and a second air flow rate; and determining a no-load limit that is lower than the first air flow rate and a loaded limit that is higher than the second air flow rate.

In yet further embodiments, the lumen of the medical device is reprocessed by flowing a reprocessing fluid into the lumen of the medical device; and flowing air through the lumen of the medical device at or below the loaded limit until substantially reaching the no-load limit. These steps may be repeated until the lumen of the medical device is reprocessed.

In other embodiments of the invention the fluidic resistances of one or more of the internal pathways of the endoscope about to be cleaned/disinfected can be determined and then compared to a database of fluidic resistances for endoscopes that have been similarly tested (e.g. ‘fluidic fingerprints’ for an endoscope model/configuration can be established), then the endoscope may be identified by matching the fluidic resistance measurements to a fluidic fingerprint in the database. In certain embodiments, such comparison can be used to confirm that the user has entered the correct endoscope information into a cleaning/disinfection device.

In one embodiment, a method of identifying a fluidic configuration of a medical device having at least one lumen comprises: determining the fluidic resistance of the at least one lumen of the medical device; and identifying the fluidic configuration based on the determined fluidic resistance of at least one lumen. In one embodiment, determining the fluidic resistance of at least one lumen of the medical device comprises: flowing a fluid comprising a known specific gravity through the at least one lumen; measuring a flow rate and/or a pressure differential of the fluid being flowed through the at least one lumen, and computing the fluidic resistance of the at least one lumen. In one aspect of the exemplary methods, identifying the fluidic configuration based on the determined fluidic resistance of the least one lumen comprises: comparing the computed fluidic resistance of the at least one lumen with a database that comprises a list of medical devices and associated fluidic resistance(s) for its respective lumen(s).

Having ascertained which lumened medical device is connected to an automated cleaning/disinfection device (by the above or other means), it is also beneficial for the device to identify which fluidic pathway of the endoscope it is connected to. An advantage of identifying the fluidic pathway/lumen can include selection of suitable cleaning/disinfection parameters (or confirming that suitable parameters have been selected) for the particular pathway of that particular endoscope configuration. For a device that cleans/disinfects multiple internal pathways of an endoscope, identification of which combination of internal pathways the device is connected to, and confirming whether device outputs are matched with the corresponding endoscope ports can aid in the selection and use of suitable cleaning/disinfection parameters for each endoscope pathway.

In the same way that the fluidic fingerprint of the entire endoscope could be matched up to a database, as described above, each lumen of an endoscope can be identified by matching its fluidic resistances with the various fluidic fingerprints for each of the lumens within a known endoscope.

In one embodiment, a method of identifying at least one lumen of a medical device comprises: determining the fluidic resistance of the at least one lumen of the medical device; and identifying the at least one lumen of the medical device based on at least its respective determined fluidic resistance. In one embodiment, determining the fluidic resistance of at least one lumen of the medical device comprises: flowing a fluid comprising a known specific gravity through the at least one lumen, measuring a flow rate and/or a pressure differential of the fluid being flowed through the at least one lumen, and computing the fluidic resistance of the at least one lumen. In another embodiment, identifying the at least one lumen of the medical device based on at least its determined fluidic resistance comprises: comparing the computed fluidic resistance with a database that comprises a list of medical device(s) and associated fluidic resistance(s) for its respective lumen(s).

Reusable medical devices/endoscopes are subject to faults due to wear and tear as well as malfunction due to general usage. Some of these faults include blockages of the internal lumens, leakage from punctures or tears of the lumens and even more subtle issues, such as partial blockages. Due to the nature of endoscopes being opaque and the long internal lumens being inside the body of the endoscope, any damage or other issues can be challenging, and in some cases impossible, to determine by simple inspection.

The inventors appreciate that many, if not all, of the aforementioned issues have in common the characteristic that they have the potential to affect the fluidic resistance of the channel. If the fluidic resistance of a channel under normal conditions is known and compared to that of the channel of an endoscope under test conditions, then a fault can be determined by this comparison. A blockage or partial blockage would result in a higher fluidic resistance than expected, whereas a leak due to a puncture or tear would result in a lower fluidic resistance than expected. This method can also be extended to determining if the connection(s) between the cleaning/disinfection device and the endoscope ports are fully engaged.

In one embodiment, a method of evaluating the integrity of a lumen of a medical device comprises: determining a fluidic resistance of the lumen; and comparing the fluidic resistance of the lumen to a known nominal range of fluidic resistance values of the lumen. In a further embodiment, determining the fluidic resistance of the lumen of the medical device comprises: flowing a fluid comprising a known specific gravity through the lumen; measuring a flow rate and/or a pressure differential of the fluid being flowed through the lumen; and computing the fluidic resistance of the lumen.

The disclosure also relates to closed-loop control systems and methods that solve problems associated with selecting suitable reprocessing fluid volumes and efficiently timing the release of that reprocessing fluid into the endoscope. For example, in some automated reprocessing devices that flow a fluid down the internal channels of an endoscope, their efficacy is tied to a combination of the volume of the fluid as well as the velocity at which the fluid travels down the lumen. As the time taken to clean/disinfect is also an important factor a balance needs to be struck. For example, for the same volume of reprocessing fluid, conveying fewer number of larger portions at or below the pressure ceiling of the endoscope will have the effect of reducing the velocity of the fluid, while conveying a greater number of smaller portions at or below the pressure ceiling of the endoscope will have the effect of increasing the velocity of the fluid but also greatly increase the time taken to clean/disinfect. The situation is further complicated in that this balance between volume, pressure, flow and time is not static, but rather can vary depending on e.g. the geometry (length, diameter etc.) of the channel the reprocessing fluid is passing through.

One solution is to test each fluidic pathway of each endoscope configuration to find the optimal settings for each and then store this information in a parameter database. This can be time consuming and updates would be required as new endoscope models are released. Additionally, there may be more subtle differences in the fluidic response of a given endoscope channel even when comparing the same channel across two endoscopes of the same model due to differences in wear & tear and even physical disposition of the endoscope in question (e.g. is the endoscope coiled or straight at the time of reprocessing). Furthermore, this approach would depend on the user entering channel and endoscope identification information into the device for each endoscope configuration, and as such would be prone to human error. Therefore, the inventors have devised an elegant method that utilizes the approach described above: running a test shot of water (or another fluid having a known specific gravity) to map out the fluidic resistance and tailor the reprocessing parameters to suit the specific endoscope channel on the fly with limited, if any, prior knowledge of the endoscope configuration.

In one embodiment, a method of reprocessing a lumen of a medical device comprises: determining a fluidic resistance of the lumen; and flowing a fluid through the lumen based on the determined fluidic resistance. In a further embodiment, determining the fluidic resistance of the lumen comprises: flowing a fluid comprising a known specific gravity through the lumen, measuring the flow rate and/or pressure differential of the fluid being flowed through the lumen, and computing the fluidic resistance of the lumen. In a yet further embodiment, flowing a fluid through the lumen based on the computed fluidic resistance comprises: irrigating the lumen with a fluid composition based on the computed fluidic resistance. In still, yet further embodiments, the method further comprises controlling at least one of: an extent of reprocessing, a volume of reprocessing fluid, a dose of reprocessing fluid, a number of shots, a timing of each shot of the number of shots, and a velocity of the fluid composition.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to.”

The above embodiments are exemplary only. Other embodiments as described herein are within the scope of the disclosed subject matter.

### BRIEF DESCRIPTION OF THE SEVERAL VIEWS OF THE DRAWINGS

So that the manner in which the features of the disclosure can be understood, a detailed description may be had by reference to certain embodiments, some of which are illustrated in the accompanying drawings. It is to be noted, however, that the drawings illustrate only certain embodiments and are therefore not to be considered limiting of its scope, for the scope of the disclosed subject matter encompasses other embodiments as well. The drawings are not necessarily to scale, emphasis generally being placed upon illustrating the features of certain embodiments. In the drawings, like numerals are used to indicate like parts throughout the various views, in which:

FIG. 1 illustrates an exemplary endoscope in accordance with one or more aspects set forth herein.

FIG. 2 shows a schematic for another embodiment of a system for the identification, evaluation, and which may be used to enable the closed-loop reprocessing of a lumen in accordance with one or more aspects set forth herein.

FIG. 3 shows a flow diagram for a method of reprocessing a lumen of a medical device in accordance with one or more aspects set forth herein.

FIG. 4 shows a flow diagram for a method of reprocessing a lumen of a medical device in accordance with certain embodiments of the disclosure.

FIG. 5 shows a flow diagram for a method of reprocessing a lumen of a medical in accordance with one or more aspects set forth herein.

FIG. 6 shows a flow diagram for a method of cleaning a lumen of a medical device in accordance with one or more aspects set forth herein.

FIG. 7A shows a schematic for one embodiment of a system for determining the reprocessing parameters which may be used to enable the closed-loop reprocessing of a lumen in accordance with one or more aspects set forth herein.

FIG. 7B shows a graph of air and water calibration data in accordance with one or more aspects set forth herein.

FIG. 7C shows a model of an air flow trace during operation of a closed-loop control system in conjunction with a reprocessing device in accordance with one or more aspects set forth herein.

FIG. 8 shows a flow diagram for a method identifying a fluidic configuration of a medical device, detecting user error, and detecting faults in accordance with certain embodiments of the disclosure.

Corresponding reference characters indicate corresponding parts throughout several views. The examples set out herein illustrate several embodiments, but should not be construed as limiting in scope in any manner.

### DETAILED DESCRIPTION

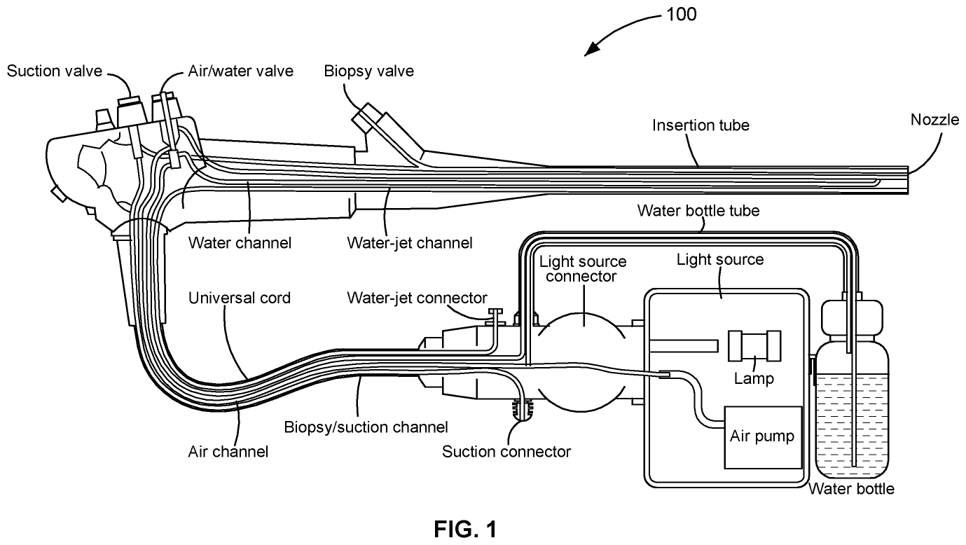

The present disclosure relates to systems and methods for the identification, evaluation, and closed-loop reprocessing of lumen(s) of medical devices, e.g., endoscope 100 (FIG. 1). Within the context of this application, references to “closed-loop reprocessing” encompass characterizing a lumen/fluidic system, and using this characterization to inform its reprocessing. One challenge is identification of the medical device, so that suitable reprocessing conditions may be employed. Methods such as selecting a device from a menu are contemplated, but may be time consuming and susceptible to user error. Additionally, they require a method of updating the menu of selections when new medical devices are released. To obviate the need to identify the medical device, the disclosure provides methods of identifying the medical device by measuring fluidic parameters, e.g., pressure, flow rate, and computing a flow coefficient.

Another form of user error occurs when the user connects the fluid source(s) to the incorrect medical device inlet. This could lead to the incorrect reprocessing parameters being applied as the reprocessing parameters suitable for the reprocessing of one channel may not be suitable if applied to another channel. Therefore, the disclosure provides methods of detecting user error by measuring fluidic parameters, e.g., pressure, flow rate, and computing a flow coefficient.

In certain aspects these fluidic parameters may be used to set closed-loop reprocessing parameters including the frequency of the delivery of apportioned amounts of fluid(s). In some embodiments, the reprocessing parameters are set once. In other embodiments, the reprocessing parameters are continuously updated based on the fluidic parameters measured in the preceding cycle.

In further aspects, a flow coefficient may be computed and used to detect user error, identify a medical device, confirm the fluidic configuration of a medical device, and/or set reprocessing parameters.

The inventors contemplate flowing a fluid (e.g., air and/or water) through the lumens of an endoscope at a set pressure and measuring the flow rate. As can be appreciated, flow rate can be kept constant, and the pressure drop measured. Additionally, both the flow rate and the pressure drop can be measured.

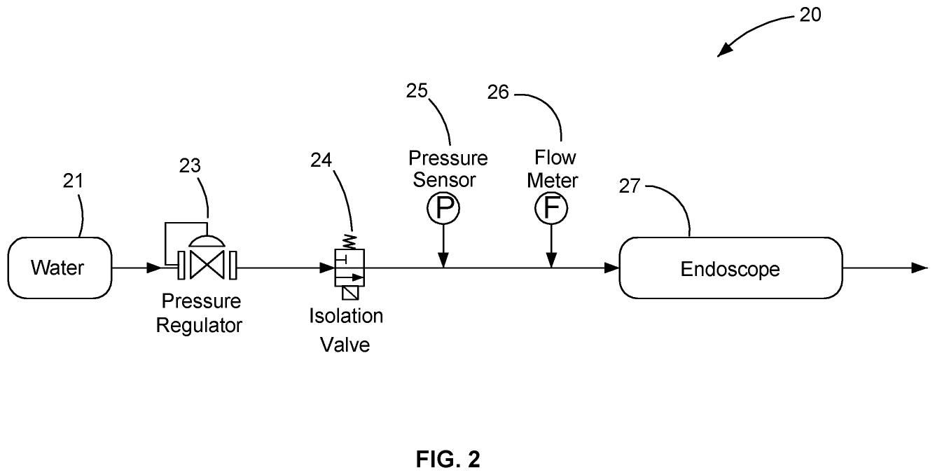

FIG. 2 shows schematic 20 for one embodiment of a closed-loop control system, in which water 21 flows through pressure regulator 23, isolation valve 24, pressure sensor 25, and flow meter 26 before flowing through endoscope 27. In this embodiment, water 21 is used as the fluid, and pressure regulator 23 is used to set the pressure of the water flow. In one embodiment pressure sensor 25 determines whether the pressure of the water is within a range of the pressure set by pressure regulator 23. In another embodiment, pressure sensor 23 is integrated with pressure regulator 23 such that pressure regulator 23 adjusts to maintain a water pressure within a range of a set pressure. It should be appreciated that isolation valve 24 may be used to stop flow to endoscope 27, e.g., before attaching/removing endoscope 27, or if the water pressure exceeds a threshold. Flow meter 26 measures the fluid flow rate of the water before it enters a lumen of endoscope 27.

In some embodiments, methods comprise measuring/determining a fluidic parameter of the at least one lumen of the medical device. Measuring/determining the fluidic parameter of at least one lumen of the medical device may comprise flowing a fluid through the at least one lumen and measuring a flow rate and/or a pressure differential of the fluid being flowed through the at least one lumen. In some embodiments, measuring/determining the fluidic parameter may comprise calculating a flow coefficient.

Reprocessing parameters may be selected based on the fluidic parameter or flow coefficient, including controlling an amount of cleaning, a volume, a dose, a number of shots, a timing of each shot of the number of shots, and a velocity of the fluid composition as described above.

In another embodiment, the pressure drop and flow rate are used to compute flow coefficient(s). In one embodiment, the computed flow coefficient(s) are used to detect user error, identify a fluidic configuration of endoscope 27, detect any fault(s) in the lumen(s) of endoscope 27, and/or set reprocessing parameters for endoscope 27.

Advantageously, lower flow coefficients correlate with higher fluidic resistance, and higher flow coefficients correlate with lower fluidic resistance. Fluidic resistance may be a function of any factor within a conduit or channel that impedes the flow of fluid, such as surface roughness or sudden bends, contractions, or expansions, and is a characteristic property of each lumen. The fluidic resistance may indicate a fluidic element or system's resistance to the flow of a given fluid through the element or system, or it may indicate the fluidic element or system's propensity to resist flow.

In addition to using air and/or water to determine the flow coefficient of at least one lumen of a medical device, any suitable fluid may be employed. Suitable fluids have a known specific gravity, so that a flow coefficient can be calculated. Exemplary fluids include gasses, such as nitrogen, argon, oxygen, ozone, and liquids including aqueous solutions, mixtures, suspensions, colloidal suspensions/dispersions, alcohols (ethanol, isopropyl alcohol), organic solvents, and combinations thereof. In some embodiments, one fluid is used to determine a first flow coefficient followed by another fluid having a different specific gravity to determine a second flow coefficient.

The flow coefficient is commonly used to evaluate the performance of a fluidic component, such as a valve. Flow coefficients are typically denoted as C; (US units) or Kr (SI units), where the value is equal to the flow across a fixed resistance for a given pressure differential. For Cᵥ, is the flow rate in US gallons per minute of water at a temperature of 60ºF with a pressure drop of 1 PSI, and for Kᵥ, it is the flow rate in m³/h of water at 16° ° C. with a pressure drop of 1 bar. In this way, flow coefficient is analogous to conductance when comparing to the electrical model, as the number is inversely proportional to the fluidic resistance. It can be appreciated that fluidic resistance describes the propensity for a fluidic system to resist flow, and one way to measure/determine fluidic resistance is to calculate a flow coefficient, which, as noted above, has an inverse relationship with fluidic resistance. The flow coefficient can be calculated by the following:

$${C}_{v} , {K}_{v} = Q \sqrt{\frac{SG}{dP}}$$

$$where :$$

$${C}_{v} = Flow coefficient ( imperial units )$$

$${K}_{v} = Flow coefficient ( metric units )$$

$$Q = Volumetric flow rate ( metric : {m}^{3} / hr , imperial : US GPM )$$

$$SG = Specific Gravity ( dimensionless )$$

$$dP = \text{} Pressure drop across \text{} fluidic component / system ( metric : bar , imperial : PSI )$$

An advantage of using flow coefficient to characterize a fluidic system is that it is easily calculated experimentally. As a known fluid (and hence known specific gravity) is pumped across a system it is relatively easy to measure the pressure drop and volumetric flow rate and therefore calculate the flow coefficient. For example, the flow coefficients of the suction biopsy and auxiliary lumens of the Olympus EVIS EXERA II CF-H180AI colonovideoscope were measured to be of the order of Kv=0.0498 and Kv=0.0063, respectively.

As the flow coefficient is an empirically generated number it can be used to predict pressures and flows within the parameters under which the number was generated. For example, the flow coefficient does not take into account the differences between laminar, transitional and turbulent flows and a value calculated in one flow regime, may not apply in another (e.g., a value calculated in a laminar flow regime may not apply in a turbulent flow regime). Similarly, as can be seen from the formula above other properties of the fluid that can influence the pressure and flow, such as viscosity, are not taken into account. Care should then be taken to ensure that the flow coefficient is calculated using measurements generated under conditions that conform to the characteristics of the flow regime and fluid properties that are of interest. If multiple, widely varying flow regimes and/or fluid properties are of interest it may be useful to calculate the flow coefficient for each of these scenarios based on measurements that are taken under corresponding conditions to avoid these inaccuracies.

There are also other formulas and approaches to characterize a fluidic system in a more comprehensive way (calculating the Reynolds Number and Darcy Friction Factor for example), which could yield another picture of how the system would respond under different flow conditions. However, these methods require information, such as lumen length and cross-sectional area, i.e., the very knowledge about the system that the methods disclosed herein seek to obtain. Moreover, these computations are rigorous and are complicated when used in connection with complex fluidic configurations. In contrast, the methods of the disclosure provide a simple approach to analyzing how restrictive a fluidic system is to flow based on easily acquired measurements. The flow coefficient is a sound approach. Of course, it can be appreciated that any technique for determining the resistance to flow in a fluidic circuit can be utilized in accordance with embodiments of the invention.

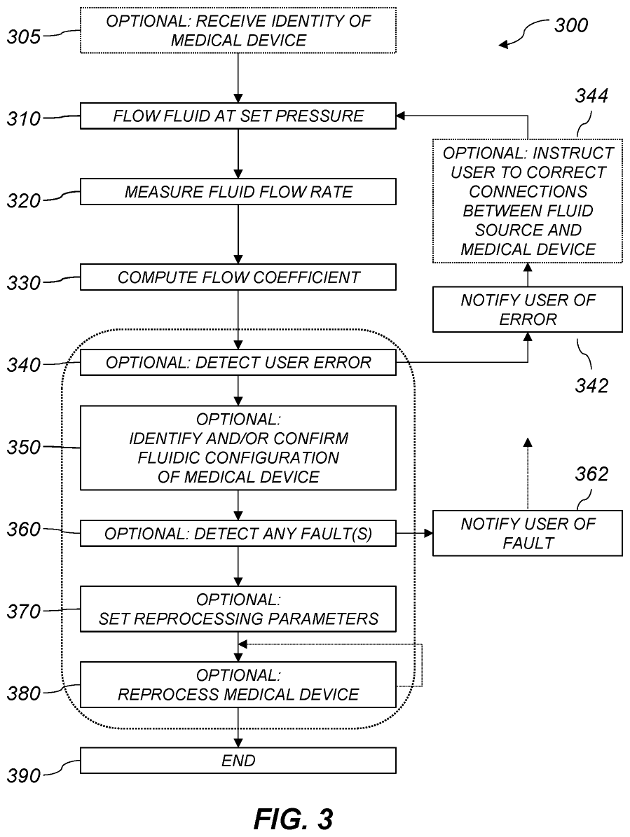

FIG. 3 shows one embodiment of a method 300 of cleaning a lumen of a medical device in accordance with embodiments of the disclosure. Optional step 305 comprises receiving the identity of a medical device. In one embodiment, the system may present a menu of user devices from which the user may select the medical device to be reprocessed, and the user's selection is received by the system. In another embodiment, the system comprises an RFID, QR code, barcode, or other identifier that the system is configured to detect and/or read, so that the system accesses and uses information regarding the medical device for use during reprocessing, e.g., the fluidic configuration and/or cleaning protocol(s). In further embodiments, optional step 305 is omitted, and the system identifies the fluidic configuration of the medical device, which advantageously avoids the possibility that the user provide the system with an incorrect medical device identity. Embodiments that identify medical devices also enable the system to detect medical devices for which there is no pre-programmed information, as would be the case for new models of medical devices having different flow characteristics.

Before or after the optional step of receiving the identity of the medical device, the user connects a fluid source to a lumen of the medical device (optional step not shown). Suitable fluids include, air, nitrogen, water, alcohol(s), cleaning fluids (e.g., a cleaning fluid comprising water, sodium bicarbonate, and/or detergent), sterilization fluids, and mixtures thereof (e.g., a 70% ethanol in water solution).

Step 310 comprises flowing fluid through the lumen at a set pressure. Suitable pressures depend on the lumen properties, e.g., lumen cross-sectional diameter and length. Many endoscopes have pressure ceilings of 24 or 26 psi, which can limit the pressures applied in the methods in accordance with embodiments of the disclosure. For example, the air pressure may be up to and including 20 psi, up to and including 21 psi, up to and including 22 psi, up to and including 23 psi, up to and including 24 psi, up to and including 25 psi, up to and including 26 psi, up to and including 27 psi, up to and including 28 psi, up to and including 29 psi, or up to and including 30 psi. In some embodiments, the air pressure is between 0.5 and 30 psi, between 10 and 30 psi, between 15 and 30 psi, between 20 and 30 psi, between 21 and 29 psi, between 22 and 28 psi, between 23 and 27 psi, or between 24 and 26 psi. In further embodiments, the air pressure is about 10 psi, about 11 psi, about 12 psi, about 13 psi, about 14 psi, about 15 psi, about 16 psi, about 17 psi, about 18 psi, about 19 psi, about 20 psi, about 20 psi, about 21 psi, about 22 psi, about 23 psi, about 24 psi, about 25 psi, about 26 psi, about 27 psi, about 28 psi, about 29 psi, or about 30 psi.

Exemplary water pressures may be up to and including 18 psi, up to and including 19 psi, up to and including 20 psi, up to and including 21 psi, up to and including 22 psi, up to and including 23 psi, up to and including 24 psi, up to and including 25 psi, up to and including 26 psi, up to and including 27 psi, or up to and including 28 psi. In some embodiments, the water pressure is between 0.5 and 28 psi, between 10 and 28 psi, between 15 and 28 psi, between 20 and 28 psi, between 21 and 29 psi, between 20 and 26 psi, between 21 and 25 psi, or between 22 and 24 psi. In further embodiments, the air pressure is about 8 psi, about 9 psi, about 10 psi, about 11 psi, about 12 psi, about 13 psi, about 14 psi, about 15 psi, about 16 psi, about 17 psi, about 18 psi, about 19 psi, about 20 psi, about 20 psi, about 21 psi, about 22 psi, about 23 psi, about 24 psi, about 25 psi, about 26 psi, about 27 psi, or about 28 psi.

Step 320 comprises measuring a fluid flow rate, which is used to compute a flow coefficient in optional step 330 as described above. It should be appreciated that any suitable flow meter or pressure sensor can be employed in the systems and methods of the disclosure. For example, without limitation, suitable flow sensors include MEMS mass flow sensors sold by Siargo Ltd. Non-limiting exemplary pressure transducers include Honeywell PX3 Series Heavy Duty Pressure Transducers. Advantageously, different pressures and flow rates can be used with the methods described herein.

The computed flow coefficient may then be employed to detect user error 340, identify the medical device 350, confirm the fluidic configuration of the medical device 350, detect any fault(s) 360, and/or set reprocessing parameters 370. Each of these optional steps is optional and may be performed in any order.

The optional step of detecting user error 340 may comprise detecting that the user has connected fluid sources to the wrong lumen and/or has failed to securely connect the fluid source to the lumen. For example, if the flow coefficient of a lumen is greater than expected, that may indicate that the lumen has been connected to a fluid source intended to couple with a lumen having a lesser flow coefficient. Conversely, if the flow coefficient of the lumen is less than expected, that may indicate that the lumen has been connected to a fluid source intended to couple with a lumen having a greater flow coefficient. A flow coefficient that is less than expected may also indicate that the connection between the fluid source and the lumen is leaking, and the user needs to more securely couple the fluid source to the lumen.

If a user error is detected, step 342 comprises notifying the user of the error. Suitable notifications include a light, a sound, text (written or auditory), an animation, or a video. Following the notification, optional step 344 comprises instructing the user how to correct connections between the fluid source and the medical device, e.g., by switching and/or tightening connections.

Optional step 350 comprises identifying and/or confirming the fluidic configuration of a medical device. Advantageously, the flow coefficients computed in optional step 330 are used to identify the fluidic configuration of the medical device. In one embodiment, the flow coefficient of a lumen (e.g., an air lumen, a water lumen, a suction lumen, a biopsy lumen, or a water-jet lumen) is matched to a flow coefficient or range of flow coefficients for a medical device to identify the medical device. In one embodiment, the flow coefficient of another lumen is matched to a flow coefficient or a range of flow coefficients for a medical device. For example, if the flow coefficient of an air lumen is matched to a medical device and the flow coefficient of a water lumen is matched to the same medical device, then confidence in the identification of the medical device is greater than the confidence in identification using the flow coefficient of one lumen. Matching of the flow coefficient(s) of additional lumens (e.g., suction and/or biopsy lumens) to known flow coefficients for a medical device is also contemplated. In one embodiment such medical device fluidic configuration identification is used to confirm the identity of the medical device entered in optional step 305.

Optional step 360 comprises detecting any faults (e.g., leaks or blockages) in the lumens of the medical device. In one embodiment, the flow coefficients computed in optional step 330 are used to faults the medical device. In one embodiment, the flow coefficient of a lumen (e.g., an air lumen, a water lumen, a suction lumen, a biopsy lumen, or a water-jet lumen) is compared to a flow coefficient or range of flow coefficients for that lumen of the medical device. In one embodiment, if the flow coefficient is greater than the flow coefficient for the lumen (or exceeds the range of acceptable flow coefficients), that would indicate that the lumen is at least partially blocked. In one embodiment, if the flow coefficient is less than the flow coefficient for the lumen (or is less than the range of acceptable flow coefficients), that would indicate that the lumen may be leaking, e.g., from a tear or puncture. The integrity of each lumen of the medical device may be determined in this manner. It should be appreciated that depending on the fluidic configuration of the medical device it may be possible to detect faults of two or more lumens in parallel.

If a fault is detected, then optional step 362 comprises notifying a user of the fault. Optional step 362 may further comprise additional optional steps, such as providing instructions for how to remedy the fault. For example, one instruction may be instructing the user to manually debride the biopsy lumen.

Optional step 370 comprises setting reprocessing parameters for the medical device. In one embodiment, the flow coefficient is used to determine reprocessing parameters, such as, the frequency of the delivery of apportioned amounts of fluid(s). For example, a lower-than-expected flow coefficient may that the channel in question is excessively soiled and would thereby indicate establishing parameters corresponding with more rigorous cleaning cycles, and a higher flow coefficient may indicate establishing parameters corresponding with less rigorous cleaning cycles. In another example, a lower-than-expected flow coefficient may indicate that the channel in question is excessively soiled and would thereby establish parameters that employ a larger volume of reprocessing fluid. In another embodiment, reprocessing parameters are updated (e.g., in ‘real-time’) as appropriate based on flow coefficient(s) to enhance cleaning efficacy.

Optional step 380 comprises reprocessing the medical device. In some embodiments the step of reprocessing employs the reprocessing parameters set in step 370. In one embodiment, the device is reprocessed by flowing a fluid comprising a cleaning agent, followed by a rinsing fluid, and sterile air to flush the rinsing fluid from each lumen of the medical device. Optional step 380 may be repeated until the cleanliness and/or sterilization of the medical device meets certain standards.

Step 390 comprises the end of the method, which may further comprise generating a report that includes the identity of the medical device, any of the measurements taken during performance of the method, any notifications generated, and/or a description or certification of the cleaning results.

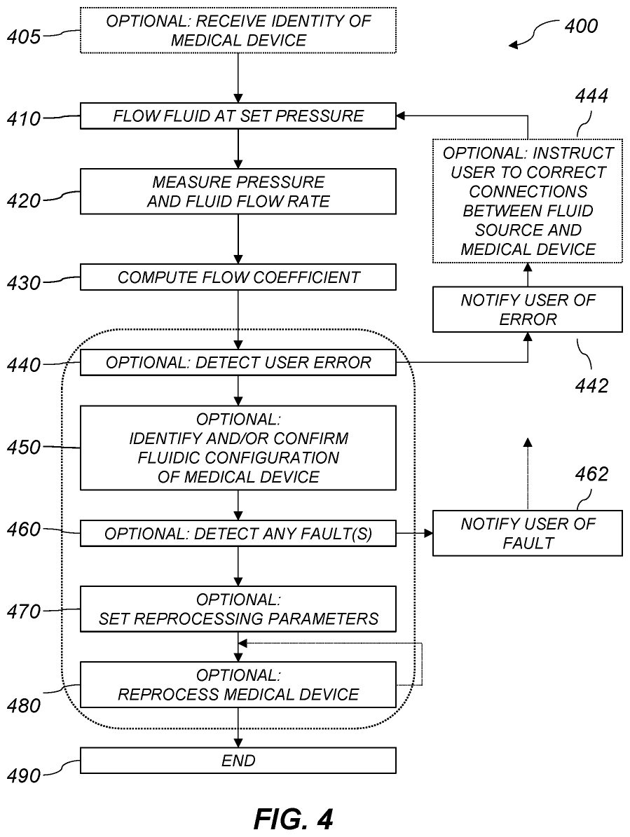

FIG. 4 shows another embodiment of a method 400 of cleaning a lumen of a medical device in accordance with embodiments of the disclosure. Optional step 405 comprises receiving the identity of a medical device. In one embodiment, the system may present a menu of user devices from which the user may select the medical device to be reprocessed, and the user's selection is received by the system. In another embodiment, the system comprises an RFID, QR code, barcode, or other identifier that the system is configured to detect and/or read, so that the system accesses and uses information regarding the medical device for use during reprocessing, e.g., the fluidic configuration and/or cleaning protocol(s). In further embodiments, optional step 405 is omitted, and the system identifies the fluidic configuration of the medical device, which advantageously avoids the possibility that the user provide the system with an incorrect medical device identity. Embodiments that identify medical devices also enable the system to detect medical devices for which there is no pre-programmed information, as would be the case for new models of medical devices having different flow characteristics.

Before or after the optional step of receiving the identity of the medical device, the user connects a fluid source to a lumen of the medical device (optional step not shown). Suitable fluids include, air, nitrogen, water, alcohol(s), cleaning fluids (e.g., a cleaning fluid comprising water, sodium bicarbonate, and/or detergent), sterilization fluids, and mixtures thereof (e.g., a 70% ethanol in water solution).

Step 410 comprises flowing fluid through the lumen at a set pressure. Suitable pressures depend on the lumen properties, e.g., lumen cross-sectional diameter and length. Many endoscopes have pressure ceilings of 24 or 26 psi, which can limit the pressures applied in the methods in accordance with embodiments of the disclosure. For example, the air pressure may be up to and including 20 psi, up to and including 21 psi, up to and including 22 psi, up to and including 23 psi, up to and including 24 psi, up to and including 25 psi, up to and including 26 psi, up to and including 27 psi, up to and including 28 psi, up to and including 29 psi, or up to and including 30 psi. In some embodiments, the air pressure is between 0.5 and 30 psi, between 10 and 30 psi, between 15 and 30 psi, between 20 and 30 psi, between 21 and 29 psi, between 22 and 28 psi, between 23 and 27 psi, or between 24 and 26 psi. In further embodiments, the air pressure is about 10 psi, about 11 psi, about 12 psi, about 13 psi, about 14 psi, about 15 psi, about 16 psi, about 17 psi, about 18 psi, about 19 psi, about 20 psi, about 20 psi, about 21 psi, about 22 psi, about 23 psi, about 24 psi, about 25 psi, about 26 psi, about 27 psi, about 28 psi, about 29 psi, or about 30 psi.

Exemplary water pressures may be up to and including 18 psi, up to and including 19 psi, up to and including 20 psi, up to and including 21 psi, up to and including 22 psi, up to and including 23 psi, up to and including 24 psi, up to and including 25 psi, up to and including 26 psi, up to and including 27 psi, or up to and including 28 psi. In some embodiments, the water pressure is between 0.5 and 28 psi, between 10 and 28 psi, between 15 and 28 psi, between 20 and 28 psi, between 21 and 29 psi, between 20 and 26 psi, between 21 and 25 psi, or between 22 and 24 psi. In further embodiments, the air pressure is about 8 psi, about 9 psi, about 10 psi, about 11 psi, about 12 psi, about 13 psi, about 14 psi, about 15 psi, about 16 psi, about 17 psi, about 18 psi, about 19 psi, about 20 psi, about 20 psi, about 21 psi, about 22 psi, about 23 psi, about 24 psi, about 25 psi, about 26 psi, about 27 psi, or about 28 psi.

Step 420 comprises measuring a pressure and a fluid flow rate, which are used to confirm that the pressure is within a certain range of the set pressure. The pressure and fluid flow rate are also used to compute a flow coefficient in step 430 as described above. Advantageously, different pressures and flow rates can be used with the methods described herein.

The flow coefficient may then be employed to detect user error 440, identify the medical device 450, confirm the fluidic configuration of the medical device 450, detect any fault(s) 460, and/or set reprocessing parameters 470. Each of these steps is optional and may be performed in any order.

The optional step of detecting user error 440 may comprise detecting that the user has connected fluid sources to the wrong lumen and/or has failed to securely connect the fluid source to the lumen. For example, if the flow coefficient of a lumen is greater than expected, that may indicate that the lumen has been connected to a fluid source intended to couple with a lumen having a lesser flow coefficient. Conversely, if the flow coefficient of the lumen is less than expected, that may indicate that the lumen has been connected to a fluid source intended to couple with a lumen having a greater flow coefficient. A flow coefficient that is less than expected may also indicate that the connection between the fluid source and the lumen is leaking, and the user needs to more securely couple the fluid source to the lumen.

If a user error is detected, step 442 comprises notifying the user of the error. Suitable notifications include a light, a sound, text (written or auditory), an animation, or a video. Following the notification, optional step 444 comprises instructing the user how to correct connections between the fluid source and the medical device, e.g., by switching and/or tightening connections.

Optional step 450 comprises identifying and/or confirming the fluidic configuration of a medical device. Advantageously, the flow coefficients computed in step 430 are used to identify the fluidic configuration of the medical device. In one embodiment, the flow coefficient of a lumen (e.g., an air lumen, a water lumen, a suction lumen, a biopsy lumen, or a water-jet lumen) is matched to a flow coefficient or range of flow coefficients for a medical device to identify the medical device. In one embodiment, the flow coefficient of another lumen is matched to a flow coefficient or a range of flow coefficients for a medical device. For example, if the flow coefficient of an air lumen is matched to a medical device and the flow coefficient of a water lumen is matched to the same medical device, then confidence in the identification of the medical device is greater than the confidence in identification using the flow coefficient of one lumen. Matching of the flow coefficient(s) of additional lumens (e.g., suction and/or biopsy lumens) to known flow coefficients for a medical device is also contemplated. In one embodiment such medical device fluidic configuration identification is used to confirm the identity of the medical device entered in optional step 405.

Optional step 460 comprises detecting any faults (e.g., leaks or blockages) in the lumens of the medical device. In one embodiment, the flow coefficients computed in step 430 are used to faults the medical device. In one embodiment, the flow coefficient of a lumen (e.g., an air lumen, a water lumen, a suction lumen, a biopsy lumen, or a water-jet lumen) is compared to a flow coefficient or range of flow coefficients for that lumen of the medical device. In one embodiment, if the flow coefficient is greater than the flow coefficient for the lumen (or exceeds the range of acceptable flow coefficients), that would indicate that the lumen is at least partially blocked. In one embodiment, if the flow coefficient is less than the flow coefficient for the lumen (or is less than the range of acceptable flow coefficients), that would indicate that the lumen may be leaking, e.g., from a tear or puncture. The integrity of each lumen of the medical device may be determined in this manner. It should be appreciated that depending on the fluidic configuration of the medical device it may be possible to detect faults of two or more lumens in parallel.

If a fault is detected, then step 462 comprises notifying a user of the fault. Step 462 may further comprise additional optional steps, such as providing instructions for how to remedy the fault. For example, one instruction may be instructing the user to manually debride the biopsy lumen.

Optional step 470 comprises setting reprocessing parameters for the medical device. In one embodiment, the flow coefficient is used to determine reprocessing parameters, such as, the frequency of the delivery of apportioned amounts of fluid(s). For example, a lower-than-expected flow coefficient may that the channel in question is excessively soiled and would thereby indicate establishing parameters corresponding with more rigorous cleaning cycles, and a higher flow coefficient may indicate establishing parameters corresponding with less rigorous cleaning cycles. In another example, a lower-than-expected flow coefficient may indicate that the channel in question is excessively soiled and would thereby establish parameters that employ a larger volume of reprocessing fluid. In another embodiment, reprocessing parameters are updated (e.g., in ‘real-time’) as appropriate based on flow coefficient(s) to enhance cleaning efficacy.

Optional step 480 comprises cleaning the medical device. In some embodiments the step of reprocessing employs the reprocessing parameters set in step 470. In one embodiment, the device is reprocessed by flowing a fluid comprising a cleaning agent, followed by a rinsing fluid, and sterile air to flush the rinsing fluid from each lumen of the medical device. Optional step 480 may be repeated until the cleanliness of the medical device meets certain standards.

Step 490 comprises the end of the method, which may further comprise generating a report that includes the identity of the medical device, any of the measurements taken during performance of the method, any notifications generated, and/or a description or certification of the cleaning results.

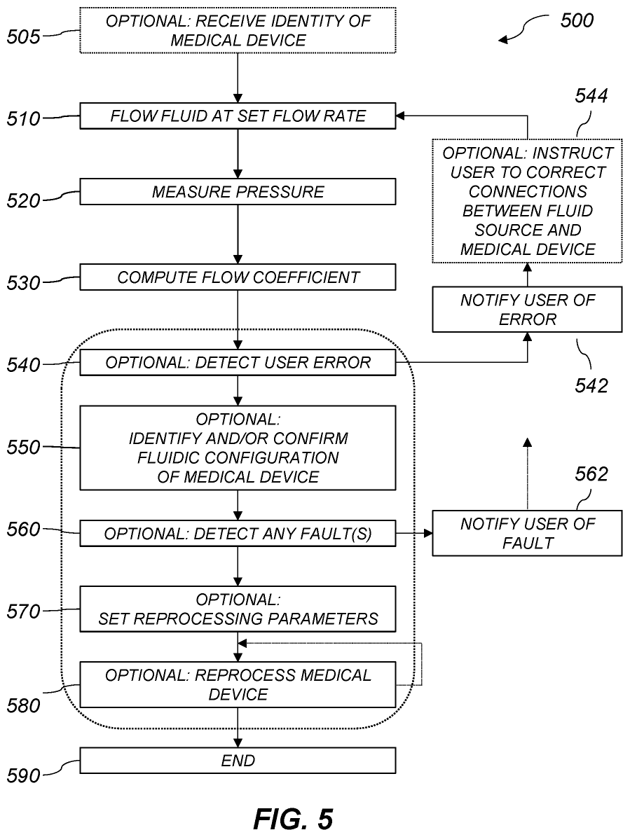

FIG. 5 shows one embodiment of a method 500 of cleaning a lumen of a medical device in accordance with embodiments of the disclosure. Optional step 505 comprises receiving the identity of a medical device. In one embodiment, the system may present a menu of user devices from which the user may select the medical device to be reprocessed, and the user's selection is received by the system. In another embodiment, the system comprises an RFID, QR code, barcode, or other identifier that the system is configured to detect and/or read, so that the system accesses and uses information regarding the medical device for use during reprocessing, e.g., the fluidic configuration and/or cleaning protocol(s). In further embodiments, optional step 505 is omitted, and the system identifies the fluidic configuration of the medical device, which advantageously avoids the possibility that the user provide the system with an incorrect medical device identity. Embodiments that identify medical devices also enable the system to detect medical devices for which there is no pre-programmed information, as would be the case for new models of medical devices having different flow characteristics.

Before or after the optional step of receiving the identity of the medical device, the user connects a fluid source to a lumen of the medical device (optional step not shown). Suitable fluids include, air, nitrogen, water, alcohol(s), cleaning fluids (e.g., a cleaning fluid comprising water, sodium bicarbonate, and/or detergent), sterilization fluids, and mixtures thereof (e.g., a 70% ethanol in water solution).

Step 510 comprises flowing fluid through the lumen at a set flow rate. Suitable flow rates depend on the lumen properties, e.g., lumen cross-sectional diameter and length. Many endoscopes have pressure ceilings of 24 or 26 psi, which can limit the pressures applied in the methods in accordance with embodiments of the disclosure. For example, air flow rates may be from about 0.1 SLPM (e.g., when the lumen has been previously filled with water) to about 5-7 SLPM (e.g., when the lumen is dry) for small diameter lumens. Exemplary air flow rates for large diameter lumens range from about 7-10 SLPM (e.g., when the lumen has been previously filled with water) to about 50 SLPM. (e.g., when the lumen is dry). When a dose of fluid (e.g., water or a cleaning fluid) is in the lumen without filling it up, then the air flow rate ranges from about 11 SLPM to about 17 SLPM.

Step 520 comprises measuring a pressure, which is used to compute a flow coefficient in step 530 as described above. Advantageously, different pressures and flow rates can be used with the methods described herein.

The flow coefficient may then be employed to detect user error 540, identify the medical device 550, confirm the fluidic configuration of the medical device 550, detect any fault(s) 560, and/or set reprocessing parameters 570. Each of these steps is optional and may be performed in any order.

The optional step of detecting user error 540 may comprise detecting that the user has connected fluid sources to the wrong lumen and/or has failed to securely connect the fluid source to the lumen. For example, if the flow coefficient of a lumen is greater than expected, that may indicate that the lumen has been connected to a fluid source intended to couple with a lumen having a lesser flow coefficient. Conversely, if the flow coefficient of the lumen is less than expected, that may indicate that the lumen has been connected to a fluid source intended to couple with a lumen having a greater flow coefficient. A flow coefficient that is less than expected may also indicate that the connection between the fluid source and the lumen is leaking, and the user needs to more securely couple the fluid source to the lumen.

If a user error is detected, step 542 comprises notifying the user of the error. Suitable notifications include a light, a sound, text (written or auditory), an animation, or a video. Following the notification, optional step 544 comprises instructing the user how to correct connections between the fluid source and the medical device, e.g., by switching and/or tightening connections.

Optional step 550 comprises identifying and/or confirming the fluidic configuration of a medical device. Advantageously, the flow coefficients computed in step 530 are used to identify the fluidic configuration of the medical device. In one embodiment, the flow coefficient of a lumen (e.g., an air lumen, a water lumen, a suction lumen, a biopsy lumen, or a water-jet lumen) is matched to a flow coefficient or range of flow coefficients for a medical device to identify the medical device. In one embodiment, the flow coefficient of another lumen is matched to a flow coefficient or a range of flow coefficients for a medical device. For example, if the flow coefficient of an air lumen is matched to a medical device and the flow coefficient of a water lumen is matched to the same medical device, then confidence in the identification of the medical device is greater than the confidence in identification using the flow coefficient of one lumen. Matching of the flow coefficient(s) of additional lumens (e.g., suction and/or biopsy lumens) to known flow coefficients for a medical device is also contemplated. In one embodiment such medical device fluidic configuration identification is used to confirm the identity of the medical device entered in optional step 505.

Optional step 560 comprises detecting any faults (e.g., leaks or blockages) in the lumens of the medical device. In one embodiment, the flow coefficients computed in step 530 are used to faults the medical device. In one embodiment, the flow coefficient of a lumen (e.g., an air lumen, a water lumen, a suction lumen, a biopsy lumen, or a water-jet lumen) is compared to a flow coefficient or range of flow coefficients for that lumen of the medical device. In one embodiment, if the flow coefficient is greater than the flow coefficient for the lumen (or exceeds the range of acceptable flow coefficients), that would indicate that the lumen is at least partially blocked. In one embodiment, if the flow coefficient is less than the flow coefficient for the lumen (or is less than the range of acceptable flow coefficients), that would indicate that the lumen may be leaking, e.g., from a tear or puncture. The integrity of each lumen of the medical device may be determined in this manner. It should be appreciated that depending on the fluidic configuration of the medical device it may be possible to detect faults of two or more lumens in parallel.

If a fault is detected, then step 562 comprises notifying a user of the fault. Step 562 may further comprise additional optional steps, such as providing instructions for how to remedy the fault. For example, one instruction may be instructing the user to manually debride the biopsy lumen.

Optional step 570 comprises setting reprocessing parameters for the medical device. In one embodiment, the flow coefficient is used to determine reprocessing parameters, such as, the frequency of the delivery of apportioned amounts of fluid(s). For example, a lower-than-expected flow coefficient may that the channel in question is excessively soiled and would thereby indicate establishing parameters corresponding with more rigorous cleaning cycles, and a higher flow coefficient may indicate establishing parameters corresponding with less rigorous cleaning cycles. In another example, a lower-than-expected flow coefficient may indicate that the channel in question is excessively soiled and would thereby establish parameters that employ a larger volume of reprocessing fluid. In another embodiment, reprocessing parameters are updated (e.g., in ‘real-time’) as appropriate based on flow coefficient(s) to enhance cleaning efficacy.

Optional step 580 comprises cleaning the medical device. In one embodiment, the device is reprocessed by flowing a fluid comprising a cleaning agent, followed by a rinsing fluid, and sterile air to flush the rinsing fluid from each lumen of the medical device. In some embodiments the step of reprocessing employs the reprocessing parameters set in step 570. Optional step 580 may be repeated until the cleanliness of the medical device meets certain standards.

Step 590 comprises the end of the method, which may further comprise generating a report that includes the identity of the medical device, any of the measurements taken during performance of the method, any notifications generated, and/or a description or certification of the cleaning results.

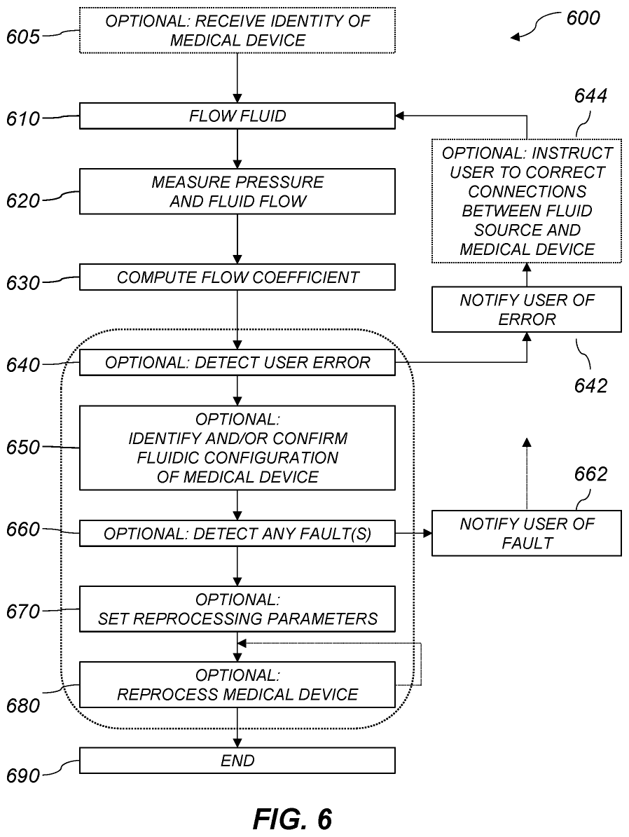

FIG. 6 shows one embodiment of a method 600 of cleaning a lumen of a medical device in accordance with embodiments of the disclosure. Optional step 605 comprises receiving the identity of a medical device. In one embodiment, the system may present a menu of user devices from which the user may select the medical device to be reprocessed, and the user's selection is received by the system. In another embodiment, the system comprises an RFID, QR code, barcode, or other identifier that the system is configured to detect and/or read, so that the system accesses and uses information regarding the medical device for use during reprocessing, e.g., the fluidic configuration and/or cleaning protocol(s). In further embodiments, optional step 605 is omitted, and the system identifies the fluidic configuration of the medical device, which advantageously avoids the possibility that the user provide the system with an incorrect medical device identity. Embodiments that identify medical devices also enable the system to detect medical devices for which there is no pre-programmed information, as would be the case for new models of medical devices having different flow characteristics.

Before or after the optional step of receiving the identity of the medical device, the user connects a fluid source to a lumen of the medical device (optional step not shown). Suitable fluids include, air, nitrogen, water, alcohol(s), cleaning fluids (e.g., a cleaning fluid comprising water, sodium bicarbonate, and/or detergent), sterilization fluids, and mixtures thereof (e.g., a 70% ethanol in water solution).

Step 610 comprises flowing fluid through the lumen without controlling the fluid pressure or flow rate.

Step 620 comprises measuring a pressure and a fluid flow rate, which are used to compute a flow coefficient in step 630 as described above. Suitable pressures and flow rates depend on the lumen properties, e.g., lumen cross-sectional diameter and length. Many endoscopes have pressure ceilings of 24 or 26 psi, which can limit the pressures applied in the methods in accordance with embodiments of the disclosure. For example, the air pressure may be up to and including 20 psi, up to and including 21 psi, up to and including 22 psi, up to and including 23 psi, up to and including 24 psi, up to and including 25 psi, up to and including 26 psi, up to and including 27 psi, up to and including 28 psi, up to and including 29 psi, or up to and including 30 psi. In some embodiments, the air pressure is between 0.5 and 30 psi, between 10 and 30 psi, between 15 and 30 psi, between 20 and 30 psi, between 21 and 29 psi, between 22 and 28 psi, between 23 and 27 psi, or between 24 and 26 psi. In further embodiments, the air pressure is about 10 psi, about 11 psi, about 12 psi, about 13 psi, about 14 psi, about 15 psi, about 16 psi, about 17 psi, about 18 psi, about 19 psi, about 20 psi, about 20 psi, about 21 psi, about 22 psi, about 23 psi, about 24 psi, about 25 psi, about 26 psi, about 27 psi, about 28 psi, about 29 psi, or about 30 psi.

Exemplary water pressures may be up to and including 18 psi, up to and including 19 psi, up to and including 20 psi, up to and including 21 psi, up to and including 22 psi, up to and including 23 psi, up to and including 24 psi, up to and including 25 psi, up to and including 26 psi, up to and including 27 psi, or up to and including 28 psi. In some embodiments, the water pressure is between 0.5 and 28 psi, between 10 and 28 psi, between 15 and 28 psi, between 20 and 28 psi, between 21 and 29 psi, between 20 and 26 psi, between 21 and 25 psi, or between 22 and 24 psi. In further embodiments, the air pressure is about 8 psi, about 9 psi, about 10 psi, about 11 psi, about 12 psi, about 13 psi, about 14 psi, about 15 psi, about 16 psi, about 17 psi, about 18 psi, about 19 psi, about 20 psi, about 20 psi, about 21 psi, about 22 psi, about 23 psi, about 24 psi, about 25 psi, about 26 psi, about 27 psi, or about 28 psi.

And suitable air flow rates include about 0.1 SLPM (e.g., when the lumen has been previously filled with water) to about 5-7 SLPM (e.g., when the lumen is dry) for small diameter lumens. Exemplary air flow rates for large diameter lumens range from about 7-10 SLPM (e.g., when the lumen has been previously filled with water) to about 50 SLPM. (e.g., when the lumen is dry). When a dose of fluid (e.g., water or a cleaning fluid) is in the lumen without filling it up, then the air flow rate ranges from about 11 SLPM to about 17 SLPM.

The flow coefficient may then be employed to detect user error 640, identify the medical device 650, confirm the fluidic configuration of the medical device 650, detect any fault(s) 660, and/or set reprocessing parameters 670. Each of these steps is optional and may be performed in any order.

For example, in one embodiment, the flow coefficient is used to detect user error 640, identify the fluidic configuration of medical device 650, confirm the fluidic configuration of the medical device 650, detect any fault(s) 660, or set reprocessing parameters 670. In another embodiment, the flow coefficient is used to detect user error 640 and identify the fluidic configuration of the medical device 650. In another embodiment, the flow coefficient is used to detect user error 640 and confirm the fluidic configuration of the medical device 650. In another embodiment, the flow coefficient is used to detect user error 640 and detect any fault(s) 660. In another embodiment, the flow coefficient is used to detect user error 640 set reprocessing parameters 670. In a further embodiment, the flow coefficient is used to identify the fluidic configuration of medical device 650 and detect any fault(s) 660. In a further embodiment, the flow coefficient is used to identify the fluidic configuration of medical device 650 and set reprocessing parameters 670. In a further embodiment, the flow coefficient is used to confirm the fluidic configuration of the medical device 650 and detect any fault(s) 660. In a further embodiment, the flow coefficient is used to confirm the fluidic configuration of the medical device 650 and set reprocessing parameters 670. In yet further embodiments, the flow coefficient is used to detect user error 640, identify the fluidic configuration of medical device 650, and detect any fault(s) 660. In yet further embodiments, the flow coefficient is used to detect user error 640, identify the fluidic configuration of medical device 650, and set reprocessing parameters 670. In this manner it should be appreciated that any of methods 300, 400, 500 or 600 may comprise any combination of detect user error 640, identify and/or confirm the fluidic configuration of the medical device 650, detect any fault(s) 660, and/or set reprocessing parameters 670, which may in turn be performed in any suitable order.

The optional step of detecting user error 640 may comprise detecting that the user has connected fluid sources to the wrong lumen and/or has failed to securely connect the fluid source to the lumen. For example, if the flow coefficient of a lumen is greater than expected, that may indicate that the lumen has been connected to a fluid source intended to couple with a lumen having a lesser flow coefficient. Conversely, if the flow coefficient of the lumen is less than expected, that may indicate that the lumen has been connected to a fluid source intended to couple with a lumen having a greater flow coefficient. A flow coefficient that is less than expected may also indicate that the connection between the fluid source and the lumen is leaking, and the user needs to more securely couple the fluid source to the lumen.

If a user error is detected, step 642 comprises notifying the user of the error. Suitable notifications include a light, a sound, text (written or auditory), an animation, or a video. Following the notification, optional step 644 comprises instructing the user how to correct connections between the fluid source and the medical device, e.g., by switching and/or tightening connections.

Optional step 650 comprises identifying and/or confirming the fluidic configuration of a medical device. Advantageously, the flow coefficients computed in step 630 are used to identify the fluidic configuration of the medical device. In one embodiment, the flow coefficient of a lumen (e.g., an air lumen, a water lumen, a suction lumen, a biopsy lumen, or a water-jet lumen) is matched to a flow coefficient or range of flow coefficients for a medical device to identify the medical device. In one embodiment, the flow coefficient of another lumen is matched to a flow coefficient or a range of flow coefficients for a medical device. For example, if the flow coefficient of an air lumen is matched to a medical device and the flow coefficient of a water lumen is matched to the same medical device, then confidence in the identification of the medical device is greater than the confidence in identification using the flow coefficient of one lumen. Matching of the flow coefficient(s) of additional lumens (e.g., suction and/or biopsy lumens) to known flow coefficients for a medical device is also contemplated. In one embodiment such medical device fluidic configuration identification is used to confirm the identity of the medical device entered in optional step 605.

Optional step 660 comprises detecting any faults (e.g., leaks or blockages) in the lumens of the medical device. In one embodiment, the flow coefficients computed in step 630 are used to faults the medical device. In one embodiment, the flow coefficient of a lumen (e.g., an air lumen, a water lumen, a suction lumen, a biopsy lumen, or a water-jet lumen) is compared to a flow coefficient or range of flow coefficients for that lumen of the medical device. In one embodiment, if the flow coefficient is greater than the flow coefficient for the lumen (or exceeds the range of acceptable flow coefficients), that would indicate that the lumen is at least partially blocked. In one embodiment, if the flow coefficient is less than the flow coefficient for the lumen (or is less than the range of acceptable flow coefficients), that would indicate that the lumen may be leaking, e.g., from a tear or puncture. The integrity of each lumen of the medical device may be determined in this manner. It should be appreciated that depending on the fluidic configuration of the medical device it may be possible to detect faults of two or more lumens in parallel.

If a fault is detected, then step 662 comprises notifying a user of the fault. Step 662 may further comprise additional optional steps, such as providing instructions for how to remedy the fault. For example, one instruction may be instructing the user to manually debride the biopsy lumen.

Optional step 670 comprises setting reprocessing parameters for the medical device. In one embodiment, the flow coefficient is used to determine reprocessing parameters, such as, the frequency of the delivery of apportioned amounts of fluid(s). For example, a lower-than-expected flow coefficient may that the channel in question is excessively soiled and would thereby indicate establishing parameters corresponding with more rigorous cleaning cycles, and a higher flow coefficient may indicate establishing parameters corresponding with less rigorous cleaning cycles. In another example, a lower-than-expected flow coefficient may indicate that the channel in question is excessively soiled and would thereby establish parameters that employ a larger volume of reprocessing fluid. In another embodiment, reprocessing parameters are updated (e.g., in ‘real-time’) as appropriate based on flow coefficient(s) to enhance reprocessing efficacy.

Optional step 680 comprises reprocessing the medical device. In one embodiment, the device is reprocessed by flowing a fluid comprising a cleaning agent, followed by a rinsing fluid, and sterile air to flush the rinsing fluid from each lumen of the medical device. In some embodiments the step of reprocessing employs the reprocessing parameters set in step 670. Optional step 680 may be repeated until the cleanliness of the medical device meets certain standards.

Step 690 comprises the end of the method, which may further comprise generating a report that includes the identity of the medical device, any of the measurements taken during performance of the method, any notifications generated, and/or a description or certification of the reprocessing results.

In some embodiments of the invention, fluidic parameters of a fluidic system are measured and used to calibrate an ensuing cleaning cycle. For example, maximum and minimum flow rates of a fluidic system can be measured, and this can be used to inform reprocessing parameters. More details around the systems/methods for reprocessing lumens using fluidic compositions comprising one or more cleaning agents can be seen in Applicant's concurrently filed patent application titled, “Systems and Methods for Cleaning Lumens with Fluidic Compositions,” which claims priority to Australian provisional patent application number 2021901729, filed Jun. 9, 2021. The contents of these applications are hereby incorporated by reference, in their entirety, especially as it relates to systems and methods for reprocessing medical devices having lumens using fluidic compositions comprising one or more cleaning agents.

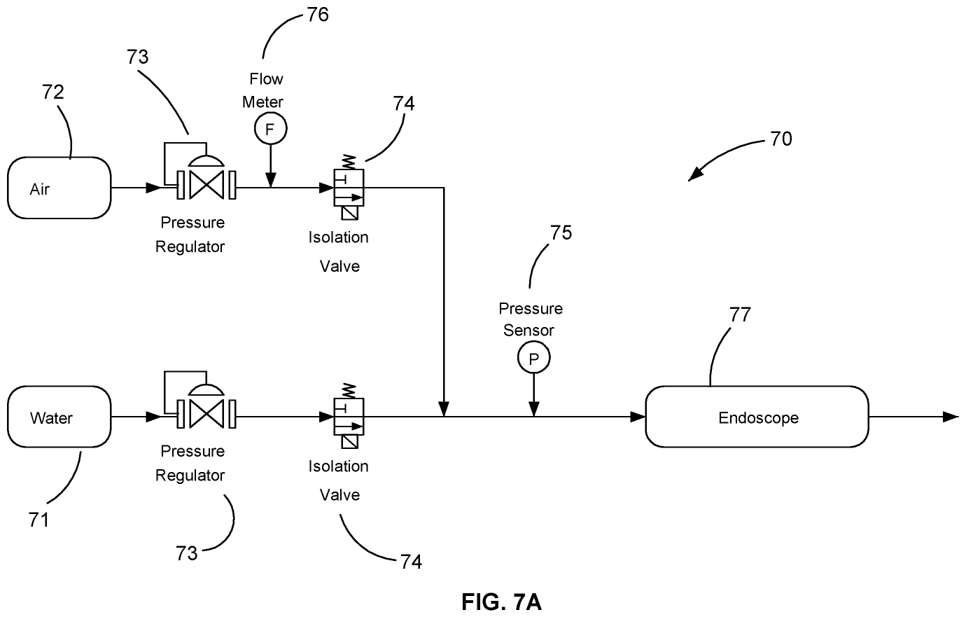

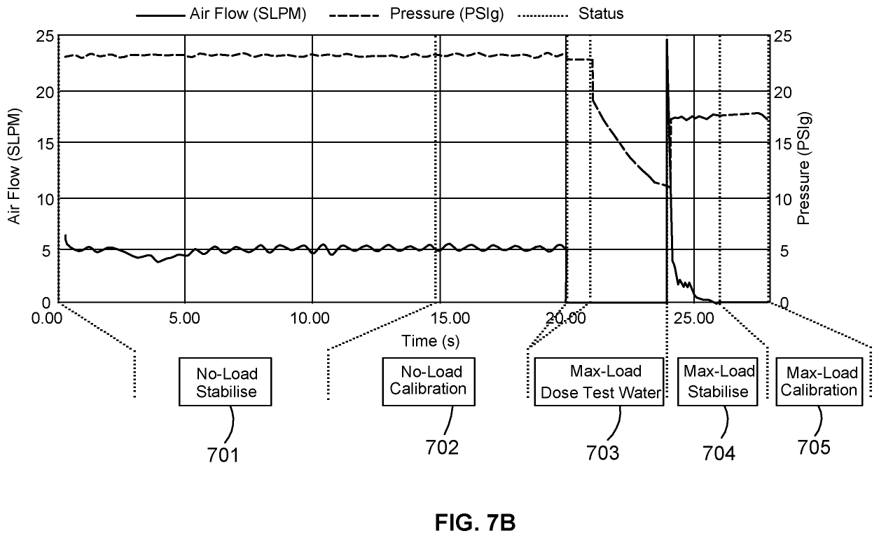

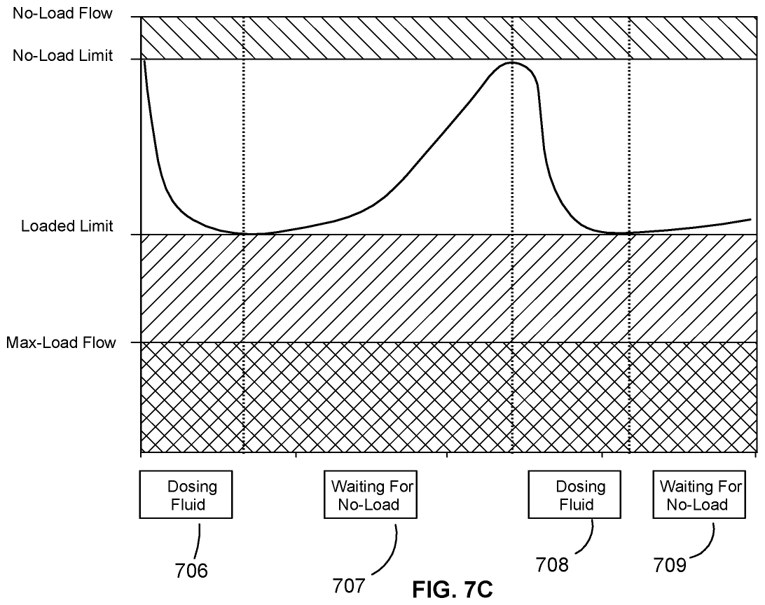

FIG. 7A shows schematic 70 for one embodiment of a closed-loop control system having air and water streams. Water 71 flows through pressure regulator 73, isolation valve 74, and pressure sensor 75 before flowing through endoscope 77. Air 72 flows through pressure regulator 73, flow meter 76, isolation valve 74, and pressure sensor 75 before flowing through endoscope 77. Isolation valves 74 may be configured to open and close depending on whether air or water should flow to the endoscope.

In one embodiment, isolation valves are configured such that the air isolation valve is open, and the water isolation valve is closed. Pressure regulator 73 regulates the pressure of air 72, and flow meter 76 measures the air flow rate. Pressure sensor 75 measures the pressure of the fluids as they enter the endoscope 77.

In one embodiment of a calibration method, isolation valves 74 are configured to such that the air isolation valve is closed, and the water isolation valve is open. Pressure regulator 73 regulates the pressure of water 71, and pressure sensor 75 measures a pressure used to monitor a dose of test water. Once the dose of test water is loaded, isolation valves 74 are switched such that water flow to endoscope 77 is stopped and air flow to endoscope 77 is started. A similar protocol may be employed during the reprocessing cycle.

FIG. 7B shows an air pressure trace and an air flow trace for the calibration method described above, and the graph is broken up into sections for each phase of calibration. The first section labelled ‘No-Load Stabilise’ 701 is where only air is conveyed down the endoscope at a set pressure (e.g., about 24 PSI) until the measurement is substantially stable. Initially, the flow data changes over time, but becomes stable within a narrow flow rate of about 5 SLPM by the end of this section 701. In the next section labelled ‘No-Load Calibration’ 702, only air continues to flow, and the control system takes these flow readings and saves them as the ‘No-Load Flow’ value. The next section labelled ‘Max-Load Dose Test Water’ 703 is where the air is turned off, and water only is pushed down the endoscope to fill it with water (or deliver a set volume of water in a “dose” or “shot”). The air flow has dropped to zero as that valve is closed, and the pressure is decaying while water enters the endoscope. In the next section labelled ‘Max-Load Stabilise’ 704, the water valve is closed when the water test shot is dosed, and the air valve is opened to push the water through the endoscope. After a lag time, the pressure and flow readings become substantially stable. In the section labelled ‘Max-Load Calibration’ 705, the water is pushed with air through the endoscope and the flow readings are saved as the ‘Max-Load Flow’ value.

Although the preceding examples use air 72 and water 71 to determine the No-Load and Max-Load parameters, such as pressure and flow rate, of at least one channel of endoscope 77, any suitable fluid may be employed. Suitable fluids include, air, nitrogen, water, alcohol(s), cleaning fluids (e.g., a cleaning fluid comprising water, sodium bicarbonate, and/or detergent), sterilization fluids, and mixtures thereof (e.g., a 70% ethanol in water solution).

Having measured and recorded the Max-Load Flow and No-Load Flow during the calibration cycle as detailed above and in the FIG. 7B, FIG. 7C models how those values may be used during a reprocessing cycle in various embodiments. The graph shows the air flow on the vertical axis with several zones. The lowest zone extends from a flow rate of zero up until the previously calibrated Max-Load Flow (the maximum load condition corresponds to the minimum flow rate). The maximum flow rate possible (for that channel) is the highest value on the graph, marked as the previously calibrated No-Load Flow. To both of these extremes buffers are applied to create the white section in the center of the graph demarked as ‘No-Load Limit’ and ‘Loaded Limit’. This defines the operating window of the reprocessing device.

In the first section of the graph, labelled as ‘Dosing Fluid’ 706, the cleaning/disinfection device is introducing some cleaning/disinfection fluid into the lumen to be cleaned/disinfected. This material slows down the flow in the channel dramatically as the air flow struggles to push the cleaning/disinfection fluid down the lumen until the flow rate hits the Loaded Limit. At this point the cleaning/disinfection device stops introducing cleaning agent into the lumen as adding any more at this stage would have the effect of slowing down the progress of the portion of cleaning/disinfection fluid as it travels through the endoscope.

In the second section, labelled as ‘Waiting for No-Load’ 707, air only continues to enter the endoscope, pushing the previously dosed cleaning/disinfection fluid down the lumen. At some point the cleaning/disinfection fluid starts to exit the endoscope and the air flow rate will increase. At some point the air flow will hit the No-Load Limit and it can be assumed that the previously dosed shot has generally exited the endoscope and thus is ready for the next shot to be dosed.

At this point the cycle repeats, Dosing Fluid 708, Waiting for No-Load 709 etc. shot after shot until the endoscope is reprocessed.

One reason for the two buffers, offsetting the No-Load Flow and the Max-Load flow are; in the first case so that the system does not have to wait for every last droplet of fluid to exit the endoscope, and in the second case to ensure that excessive cleaning/disinfection fluid is not delivered to the endoscope for that shot.

Returning to the concept of fluidic resistance, because fluidic resistance is a property of the lumen, it may be used to identify the fluidic configuration of an endoscope and/or detect any fault(s) in the lumen(s) of the endoscope. Mapping out the fluidic resistances of the various internal fluidic pathways of an endoscope could be beneficial in the following applications.

In one embodiment, a method of identifying at least one lumen of a medical device comprises: determining the fluidic resistance of the at least one lumen of the medical device; and identifying the at least one lumen of the medical device based on at least its respective determined fluidic resistance.

In one embodiment, determining the fluidic resistance of at least one lumen of the medical device comprises: flowing a fluid comprising a known specific gravity through the at least one lumen, measuring a flow rate and/or a pressure differential of the fluid being flowed through the at least one lumen, and computing the fluidic resistance of the at least one lumen.

In another embodiment, identifying the at least one lumen of the medical device based on at least its determined fluidic resistance comprises: comparing the computed fluidic resistance with a database that comprises a list of medical device(s) and associated fluidic resistance(s) for its respective lumen(s).

In one embodiment, a method of identifying a fluidic configuration of a medical device having at least one lumen comprises: determining the fluidic resistance of the at least one lumen of the medical device; and identifying the fluidic configuration based on the determined fluidic resistance of at least one lumen. In one embodiment, determining the fluidic resistance of at least one lumen of the medical device comprises: flowing a fluid through the at least one lumen with a fluid having a known specific gravity; measuring a flow rate and/or a pressure differential of the fluid being flowed through the at least one lumen, and computing the fluidic resistance of the at least one lumen.

In one aspect of the exemplary methods, identifying the fluidic configuration based on the determined fluidic resistance of the least one lumen comprises: comparing the computed fluidic resistance of the at least one lumen with a database that comprises a list of medical devices and associated fluidic resistance(s) for its respective lumen(s).

In one embodiment, a method of evaluating the integrity of a lumen of a medical device comprises: determining a fluidic resistance of the lumen; and comparing the fluidic resistance of the lumen to a known nominal range of fluidic resistance values of the lumen. In another embodiment, determining the fluidic resistance of the lumen of the medical device comprises: flowing a fluid comprising a known specific gravity through the lumen; measuring a flow rate and/or a pressure differential of the fluid being flowed through the lumen; and computing the fluidic resistance of the lumen.

In one embodiment, a method of reprocessing a lumen of a medical device comprises: determining a fluidic resistance of the lumen; and flowing a fluid through the lumen based on the determined fluidic resistance. In another embodiment, determining the fluidic resistance of the lumen comprises: flowing a fluid comprising a known specific gravity through the lumen, measuring the flow rate and/or pressure differential of the fluid being flowed through the lumen, and computing the fluidic resistance of the lumen. In a further embodiment, flowing a fluid through the lumen based on the computed fluidic resistance comprises: irrigating the lumen with a fluid composition based on the computed fluidic resistance. In yet further embodiments, the method further comprises controlling at least one of: an extent of cleaning, a volume of cleaning fluid, a dose of cleaning fluid, a number of shots, a timing of each shot of the number of shots, and a velocity of the fluid composition.

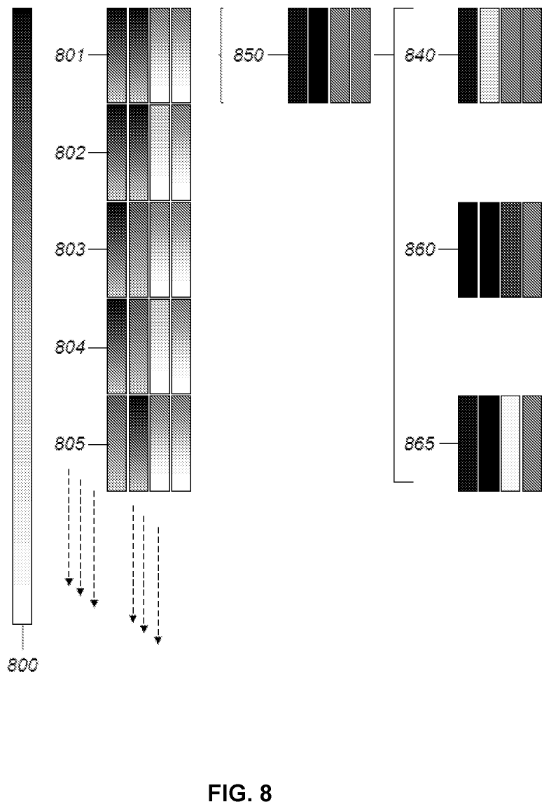

FIG. 8 is a schematic representation of one embodiment of detecting user error 840, confirming the fluidic configuration of the medical device 850, and detecting faults 860 and 865. Fluidic resistance scale 800 provides a grayscale representation of potential fluidic resistances, where lighter shades indicate lower fluidic resistance, and darker shades indicate higher fluidic resistance. Different medical devices have fluidic resistance finger prints 801, 802, 803, 804, 805, may continue for further medical devices as indicated by the dashed arrows. Each fingerprint includes a grayscale representation of the fluidic resistance ranges expected for each lumen of the corresponding medical device. Computed fluidic resistances for each channel of a medical device are used to generate fingerprint 850, which matches fingerprint 801 and identifies/confirms that the medical device having fingerprint 850 is a medical device of the same make and model of the medical device having fingerprint 801. For the medical device identified using fingerprint 850, fingerprint 840 illustrates user error. The lower fluidic resistance in the second lumen indicates that the fluid connector is leaking and needs to be secured. Fingerprint 860 illustrates a blockage in the third lumen of the medical device, because the fluidic resistance is higher than the expected range. Fingerprint 865 illustrates a puncture in the third lumen of the medical device, because the fluidic resistance is lower than the expected range.

It should be appreciated that the closed-loop control systems described herein may be used in conjunction with any suitable reprocessing device. For example, Applicants have proposed systems and methods for reprocessing a medical device having a lumen using cleaning agent fluidic compositions, and these may be amenable to the disclosed techniques. In general, such systems and methods include: creating/obtaining a liquid-powder mixture that is fluidic; apportioning the liquid-powder mixture into a suitable amount; and delivering the apportioned amount through at least a portion of a lumen/channel to be reprocessed. Thus, for instance, the techniques disclosed herein may be used to control the apportionment of the liquid-powder mixture and/or the delivering of the apportioned amount. For example, the disclosed techniques may be used to determine that the suction/biopsy channel of an endoscope is to be the target of reprocessing. Accordingly, as suction/biopsy channels are relatively larger, this information may be used to establish a relatively larger size of the apportioned amount. Conversely, where it is determined that air-water channels are the target of reprocessing, this information can be used to establish a relatively smaller size of the apportioned amount.

Moreover, the techniques disclosed herein can continually be used to determine the fluidic resistance and update reprocessing parameters (e.g., in ‘real-time’) as appropriate to enhance reprocessing efficacy. For example, the frequency of the delivery of apportioned amounts can be informed by the determined fluidic resistance. More details around the systems/methods for reprocessing lumens using fluidic compositions comprising one or more cleaning agents can be seen in Australian provisional patent application number 2021901729, filed Jun. 9, 2021. The contents of this application is hereby incorporated by reference, in their entirety, especially as it relates to systems and methods for reprocessing medical devices having lumens using fluidic compositions comprising one or more cleaning agents.

Of course, it is to be appreciated that the techniques disclosed herein can find applicability in any of a variety of systems. For example, Applicants have additionally proposed “synergistic cleaning systems and methods for medical devices having a lumen.” Such systems and methods generally involve: delivering a target dosage of a cleaning agent to an eductor; optionally delivering a surfactant to the eductor; delivering a liquid to the eductor to create a mixture of cleaning agent, liquid, and optionally a surfactant; and delivering the mixture to a target lumen to be reprocessed using a carrier fluid. More details around the synergistic reprocessing systems/methods for medical devices having lumens can be seen in Australian provisional patent application number 2021901732, filed Jun. 9, 2021, and titled: “Synergistic Cleaning Systems and Methods for Medical Devices Having a Lumen.” The contents of “Synergistic Cleaning Systems and Methods for Medical Devices Having a Lumen” are hereby incorporated by reference, in their entirety, especially as it relates to synergistic reprocessing systems/methods for medical devices having a lumen.

1-24. (canceled)

25. A method of reprocessing a lumen of a medical device comprising:
measuring at least one fluidic parameter of the at least one lumen of the medical device; and
reprocessing the lumen of the medical device based on the at least one measured fluidic parameter of the at least one lumen.

26. The method of claim 25, wherein measuring the at least one fluidic parameter of the at least one lumen comprises:
flowing a fluid through the at least one lumen,
measuring at least one of a flow rate or a pressure differential of the fluid being flowed through the at least one lumen.

27. The method of claim 26, wherein reprocessing the at least one lumen of the medical device based on the at least one measured fluidic parameter comprises:
comparing the at least one of the flow rate and the pressure differential of the at least one lumen with a database that comprises a list of medical devices and associated one or more flow rates or pressure differential for its respective one or more lumens.

28. The method of claim 26, wherein reprocessing the at least one lumen of the medical device based on the at least one measured fluidic parameter of at least one lumen comprises:
computing a flow coefficient of the at least one lumen from the at least one of the flow rate or the pressure differential, and
comparing the computed flow coefficient of the at least one lumen with a database that comprises a list of medical devices and associated one or more flow coefficients for its respective one or more lumens.

29-32. (canceled)

33. The method of claim 26, wherein measuring at least one of the flow rate or the pressure differential of the fluid being flowed through the at least one lumen comprises:
measuring the flow rate and the pressure differential of the fluid being flowed through the at least one lumen.

34. The method of claim 25, wherein the reprocessing the lumen of the medical device based on the at least one measured fluid parameter of the at least one lumen comprises:
obtaining a liquid-powder mixture that is fluidic;
apportioning the liquid-powder mixture into one or more apportioned amounts; and
delivering each the one or more apportioned amounts through at least a portion of the lumen.

35. The method of claim 34, wherein the method further comprises:
determining the one or more apportioned amounts based on the at least one measured fluid parameter of the at least one lumen.

36. The method of claim 34, wherein the one or more apportioned amounts are delivered to the at least one lumen with a velocity, and wherein the method comprises:
determining the velocity based on the at least one measured fluid parameter of the at least one lumen.

37. The method of claim 34, wherein the one or more apportioned amounts comprises a plurality of apportioned amounts that are delivered to the at least one lumen, and wherein the method comprises:
determining a timing of the delivery of the plurality of apportioned amounts based on the at least one measured fluid parameter of the at least one lumen.

38. The method of claim 34, wherein the one or more apportioned amounts comprises a selected number of apportioned amounts that are delivered to the at least one lumen, and wherein the method comprises:
determining the selected number of apportioned amounts based on the at least one measured fluid parameter of the at least one lumen.

39. The method of claim 25, further comprising:
determining the selected number of apportioned amounts based on the at least one measured fluid parameter of the at least one lumen.

40. The method of claim 25, wherein measuring the fluidic parameter of the at least one lumen comprises:
determining a fluidic resistance of the at least one lumen.

41. The method of claim 40, wherein the method further comprises:
identifying a fluidic configuration of the at least one lumen based on the determined fluidic resistance of at least one lumen.

42. The method of claim 40, wherein determining the fluidic resistance of at least one lumen comprises:
flowing a fluid comprising a known specific gravity through the at least one lumen;
measuring at least one of flow rate or a pressure differential of the fluid being flowed through the at least one lumen; and
computing the fluidic resistance of the at least one lumen.

43. The method of claim 40, wherein the method further comprises:
identifying the at least one lumen of the medical device based on at least its respective determined fluidic resistance.

44. The method of claim 40, further comprising:
evaluating an integrity of the at least one lumen based on the fluidic resistance of the lumen.

45. The method of claim 44, wherein evaluating the integrity of the at least one lumen comprises:
comparing the fluidic resistance of the at least one lumen to a known nominal range of fluidic resistance values of the at least one lumen.
